(19) United States
(12) Patent Application Publication
(10) Pub. No.: US 2025/0270154 A1
(43) Pub. Date: Aug. 28, 2025

(54) PROCESS FOR SEPARATING PARAFFINS

(71) Applicant: UOP LLC, Des Plaines, IL (US)

(72) Inventor: Scott Lyle Nauert, Chicago, IL (US)

(21) Appl. No.: 19/050,978

(22) Filed: Feb. 11, 2025

Related U.S. Application Data
(60) Provisional application No. 63/557,295, filed on Feb. 23, 2024.

(51) Int. Cl. C07C 7/04; C07C 7/00; C07C 7/09

(52) U.S. Cl. CPC C07C 7/04; C07C 7/005; C07C 7/09

(57) ABSTRACT

A process for separating paraffins is disclosed. The process comprises separating a paraffinic stream to provide a first stream comprising C3− hydrocarbons and a second stream comprising C3+ hydrocarbons. The first stream is compressed to provide a first compressed stream. The first compressed stream is fractionated in a fractionation column to provide an overhead stream and a bottom stream comprising C2+ hydrocarbons.

### FIELD

The field is the separation of light paraffins. The field may particularly relate to separation of paraffins from a naphtha to light paraffinic reactor effluent stream.

### BACKGROUND

Light olefin production is vital to the production of sufficient plastics to meet worldwide demand. Dehydrogenation is a process in which light paraffins such as ethane and propane can be dehydrogenated to make ethylene and propylene, respectively typically in the presence of a catalyst. Dehydrogenation can be achieved in either the presence of an oxidant such as oxygen or in the absence of an oxidant. Non-oxidative dehydrogenation is an endothermic reaction which requires external heat to drive the reaction to completion. Propane dehydrogenation (PDH) is a widely practiced example of non-oxidative dehydrogenation to produce propylene from propane. Ethane oxidative dehydrogenation is a newer oxidative process for converting ethane to ethylene which can be conducted at lower temperatures with lower carbon oxide emissions than non-oxidative and thermal cracking processes.

Fluid catalytic cracking (FCC) is another endothermic process that can be tuned to produce substantial propylene. However, not every FCC unit is tuned to make substantial propylene. Also, high propylene FCC units do not recover much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to a cracking furnace to reduce the hydrocarbon partial pressure and enhance olefin yield and to reduce the formation and deposition of carbonaceous material in the cracking reactors. The process is therefore often referred to as steam cracking or pyrolysis.

Paraffins with a range of carbon numbers can be thermally cracked to produce olefins including ethane, propane, butanes, and naphtha. Ethane and naphtha feeds are typical due to higher light olefin yield than propane and butane feeds. Ethane feed is used in regions where light hydrocarbon gases are prevalent. In regions where gas is not abundant, naphtha feed is employed for steam cracking. Naphtha steam cracking has long set the price in the ethylene industry due to higher production cost versus ethane steam cracking. The world does not currently produce enough ethane to supply the growing demand for ethylene. Therefore, regions lacking ethane supply such as Asia and Europe rely mainly on naphtha steam cracking to supply ethylene. Naphtha steam cracking yields only about 30%-35% ethylene with the balance including both relatively high-value by-products comprising propylene, butadiene, and butene-1 and relatively low value by-products comprising pyoil, pygas, and fuel gas. Additional pressures on naphtha steam cracking including minimum production requirements and environmental concerns have led to the withholding of government approvals in certain regions such as China. The ethylene industry needs a more efficient, economical and environmentally friendly route to light olefins from naphtha feeds.

### BRIEF SUMMARY

A process for separating paraffins is disclosed. The process comprises separating a paraffinic stream to provide a first overhead stream comprising C3− hydrocarbons and a second stream comprising C3+ hydrocarbons. The first stream may be compressed to provide a first compressed stream. The first compressed stream is fractionated in a fractionation column to provide an overhead stream and a bottom stream comprising C2+ hydrocarbons. The present process for separating paraffins minimizes the utility duties in the column condensers and the column reboilers for separation.

### BRIEF DESCRIPTION OF THE DRAWINGS

FIG. 1 is a schematic drawing of an exemplary embodiment of a paraffins separation process and apparatus of the present disclosure.

FIG. 2 is a schematic drawing of another exemplary embodiment of the paraffins separation process and apparatus of the present disclosure.

FIG. 3 is a schematic drawing of yet another exemplary embodiment of the paraffins separation process and apparatus of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx−” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take the main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

### DETAILED DESCRIPTION

Naphtha and liquefied petroleum gas (LPG) feed stock comprising C3-C8 hydrocarbons is primarily charged to a “Naphtha to Ethane and Propane” (NEP) reactor to convert naphtha and LPG in the presence of hydrogen into desirable ethane and propane along with less desirable methane. Separation of the light paraffins from the reactor effluent may require higher utility duties in the fractionation columns and higher reboiler temperatures which require more expensive utilities. The disclosed process provides a heat integration between the fractionation columns and process streams in the NEP unit and enables using higher-temperature refrigerants for cooling some of the streams.

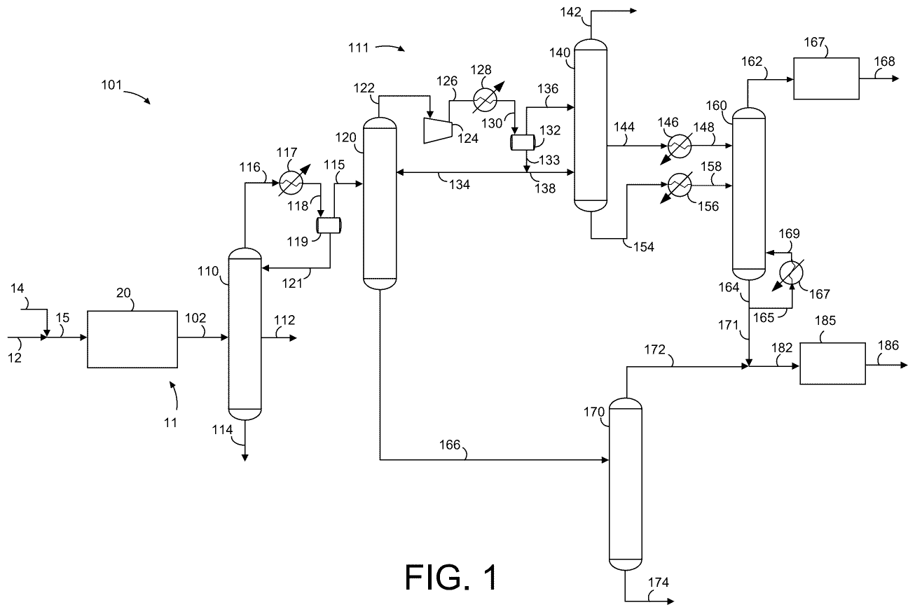

Turning to FIG. 1, an embodiment of a process and an apparatus for separating paraffins 101 is disclosed. The process comprises a naphtha to ethane and propane (NEP) reactor section 11 and a separation unit 111. The NEP reactor section 11 may comprise a NEP reactor 20 and a splitter column 110. A naphtha stream in line 12 may be combined with a hydrogen stream in line 14 to provide a charge stream in line 15. The charge stream in line 15 may be heated and charged to a naphtha to ethane and propane (NEP) reactor 20 to be contacted with an NEP catalyst. In an aspect, a heavy stream may be combined with the naphtha stream in line 12. The naphtha stream may comprise C4 to C12 hydrocarbons preferably having a T10 between about-10° C. and about 60° C. and a T90 between about 70 and about 180° C. The naphtha feed stream may comprise normal paraffins, iso-paraffins, naphthenes, and aromatics. The naphtha stream may be heated to a reaction temperature of about 300° C. to about 600° C., suitably between about 325° C. and about 550° C., and preferably between about 350° C. and about 525° C. Weight space velocity should be between about 0.3 to about 20 hr⁻¹, suitably between about 0.5 and about 10 hr⁻¹ and preferably between about 1 to about 4 hr⁻¹. A total pressure should be about 0.1 to about 3 MPa (abs), suitably no less than about 1 MPa (abs) and preferably from about 1.5 to about 2.5 MPa (abs). At these conditions, C2-C4 paraffinic yield is consistently in an excess of 80 wt %, while methane yield is less than about 16 wt %, suitably below about 14 wt % and typically below about 12 wt % and preferably no more than 10 wt %. Under these conditions, ethane can make up more than 60 wt % of the total C2 to C3 and for that matter C2 to C4 produced in the NEP reactor 20.

The hydrogen-to-hydrocarbon molar ratio is important to producing ethane and propane. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2. Low hydrogen-to-hydrocarbon ratio promotes desired reaction kinetics which are initiated with dehydrogenation. Hydrogen-to-hydrocarbon ratio may range from about 50% to about 500%, suitably no more than 300% and preferably no more than 200% of stoichiometric requirements to convert naphtha molecules to ethane and/or propane. The molar ratio of hydrogen to hydrocarbon depends on the feed type including paraffin, olefin, naphthene or aromatics, the feed molecular carbon number, and the desired product between predominantly ethane, predominantly propane or ethane and propane of comparable abundance.

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙAlₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 20 to 400, 20 to 200 or 20 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce 1/888-inch to about 1/32-inch diameter calcined supports. Alternatively, the zeolite may be mixed with a silicon oxide containing binder and extruded to 1/32 to ¼-inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst is steam oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 500 to about 550° C. for 3 hours before contacting feed.

After paraffin conversion, a reactor effluent stream comprising paraffins is discharged from the NEP reactor 20 in an effluent line 102. The reactor effluent stream in line 102 may be a light paraffinic stream. The reactor effluent stream in line 102 may comprise at least about 40 wt % ethane or at least about 40 wt % propane or at least about 70 wt % and preferably at least about 80 wt % ethane and propane. The ethane to propane ratio can range from about 0.1 to about 5. The reactor effluent stream in line 102 can have less than about 16 wt %, suitably less than about 14 wt %, preferably less than about 12 wt %, and more preferably less than about 10 wt % methane.

The reactor effluent stream in line 102 may be cooled and fed to a splitter column 110 to separate aromatics in a splitter heavy stream. The splitter heavy stream is taken from the splitter column 110 in a bottoms line 114 and may be rich in aromatics. In an embodiment, the splitter heavy stream in line 114 may be recycled to the NEP reactor in charge line 15.

A splitter light stream rich in paraffins is produced in an overhead line 116 of the splitter column 110. A splitter light stream in line 116 may be passed to the separation unit 111 to separate one or more paraffins from the splitter light stream in line 116. In an embodiment, a splitter side stream may be provided in line 112 from the splitter column 110. In this embodiment, the splitter side stream in line 112 may comprise single ring aromatics that can be recycled to the NEP reactor in charge line 15. Moreover, in this embodiment, multi-ring aromatics may be produced in the splitter bottoms line 114.

In an exemplary embodiment, the splitter column 110 may comprise two reboilers, an upper reboiler 113 and a lower reboiler 197. The splitter side stream in line 112 may be collected from a tray below the feed stage for the reactor effluent stream in line 102. In an aspect, all the liquid may be collected in the splitter side stream in line 112 from the tray below the feed stage. The splitter side stream in line 112 may be heated in the upper reboiler 113 to provide an upper reboiled stream in line 191. The upper reboiled stream in line 191 may be recycled back to the splitter column 110 below the draw stage for the splitter side stream in line 112. In an embodiment, a portion of the upper reboiled stream in line 191 may be taken in line 192 from the splitter column 110. The remaining portion of the upper reboiled stream in line 191 may be recycled in line 193 back to the splitter column 110 below the draw stage.

A lower reboiling stream may be taken in lower reboiling line 195 from the splitter heavy stream in line 114. The lower reboiling stream in line 195 may be heated in the lower reboiler 197 to provide a lower reboiled stream in line 198 which is recyled back to the splitter column 110. The remaining portion of the splitter heavy stream is taken as net liquid bottoms stream in line 194 from the bottom of the splitter column 110. In an embodiment, the splitter light stream which may be in the splitter overhead line 116 may be separated into vapor and liquid streams before passing it to the separation unit 111 to separate paraffins. The splitter light stream in line 116 may be cooled in a heat exchanger 117 to provide a cooled splitter light stream in line 118. The cooled splitter light stream in line 118 is passed to a splitter receiver 119 in which vapor and liquid are separated. A splitter net overhead vapor stream is provided in line 115 from the splitter receiver 119. The splitter net overhead vapor stream in line 115 is passed to the separation unit 111. A splitter overhead liquid stream is produced in line 121 from the splitter receiver 119 and passed to the splitter column 110 as a reflux stream.

The NEP separation unit 111 may be a separator and a fractionation column or a series of fractionation columns and other separation units. The NEP separation unit 111 may comprise a separator 120 and a fractionation column 140. In an embodiment, the separator 120 may be a fractionation column. In this embodiment, the NEP separation unit 111 may comprise a first fractionation column 120 and a second fractionation column 140. In an exemplary embodiment, the first fractionation column may be a light ends splitter column 120 which may be a first depropanizer column. In another exemplary embodiment, the second fractionation column 140 may be a demethanizer column 140.

The current process comprises a light ends splitter column 120 upstream of the demethanizer column 140 in the separation unit 111. The upstream light ends splitter column prevents most of the C4+ heavy hydrocarbons from flowing to the demethanizer column 140 and/or a deethanizer column 160. Otherwise, the presence of C4+ heavy hydrocarbons may lead to higher utility duties in the fractionation columns and higher reboiler temperatures which require more expensive utilities. In addition, C4+ hydrocarbons may have higher viscosity which can reduce heat exchanger performance in the demethanizer column 140 and the deethanizer column 160 and result in more frequent cleaning. In some cases, the presence of significant benzene can lead to freezing in the cold demethanizer column 140. The current process prevents this by removing C4+ hydrocarbons in the bottoms of the light ends splitter column 120.

Referring to the separation unit 111, the splitter net overhead vapor stream in line 115 is passed to the light ends splitter column 120. C1-C4 hydrocarbons may be separated in the light ends splitter column 120. A first overhead stream comprising C3− hydrocarbons may be produced from an overhead in a total overhead line 122 from the light ends splitter column 120. A second stream comprising C3+ hydrocarbons may be produced from a bottom in a bottoms line 166 from the light ends splitter column 120. More than about 99 mol % of C2− hydrocarbons may be recovered in the light ends splitter overhead stream of the light ends splitter column overhead line 136, and more than about 95 mol % of C4+ hydrocarbons may be recovered in the light ends splitter bottoms stream in the light ends splitter bottoms line 166. The light ends splitter column 120 may be operated at an overhead pressure of about 1034 kPa (gauge) (150 psig) to about 2068 kPa (gauge) (300 psig), and a bottoms temperature of about 80° C. (176° F.) to about 160° C. (320° F.).

The first stream comprising C3− hydrocarbons in the light ends splitter total overhead line 122 is heat integrated with a compressor 124. The first stream in the light ends splitter total overhead line 122 may be compressed in the light ends compressor 124 to provide a first compressed stream in line 126. In an exemplary embodiment, the first stream in the light ends splitter total overhead line 122 may be compressed to a pressure of about 1724 kPa (gauge) (250 psig) to about 3792 kPa (gauge) (550 psig) in the light ends compressor 124. In an embodiment, the first stream in the light ends splitter total overhead line 122 is compressed to a pressure of at least about 2.1 MPa (g) (300 psig) or at least about 2.8 MPa (g) (400 psig) or at least about 3.1 MPa (g) (450 psig) to provide a first compressed stream in line 126. The first compressed stream in line 126 may be passed to a light ends splitter condenser 128 to partially condense the first compressed stream in line 126 and provide a first cooled stream in line 130. The light ends splitter condenser 128 may comprise one or more heat exchangers. Suitable heat exchange media for condensing the first compressed stream in line 126 in the light ends splitter condenser 128 include air, cooling water, refrigerants such as propane or propylene, and other process streams. The first cooled stream in line 130 may be passed to a light ends splitter receiver 132 for separation into a first light ends net overhead vapor stream in line 136 and a first light ends overhead liquid stream in line 133. A reflux stream is taken in line 134 from the first light ends overhead liquid stream in line 133 and passed to the light ends splitter column 120. A net first light ends overhead liquid stream may be taken in line 138 from the first light ends overhead liquid stream in line 133.

The overhead compressor 124 for compressing the first stream in the total light ends splitter overhead line 122 serves two benefits: 1) the vapor needs to be compressed to a higher pressure such as about greater than 400 psig for separation in the demethanizer column 140, and 2) condensing the first stream in the total light ends splitter overhead line 122 at higher pressure increases the temperature at which further separation is conducted. This enables heat integration between the condenser and other process streams in the process and enables use of higher temperature refrigerants which are more economical than the usual lower temperature refrigerants.

When desired to recover propane as a product stream, more than about 90 mol % of propane may be recovered in the first light ends net overhead vapor stream in line 136 and/or the net first light ends overhead liquid stream in line 138. When desired to recycle propane to the NEP reactor, then more than about 50 mol % of propane may be recovered in the second stream in the light ends splitter bottoms line 166, preferably more than about 75 mol % propane may be recovered in the second stream in the light ends splitter bottoms line 166, and more preferably more than about 90 mol % propane may be recovered in the second stream in the light ends splitter column bottom line 166.

The first light ends net overhead vapor stream in line 136 is a high-pressure vapor stream which has a lower concentration of C4+ hydrocarbons. The first light ends net overhead vapor stream in line 136 may be separated in the demethanizer column 140 and optionally a deethanizer column 160 to produce a net gas stream comprising hydrogen and methane, an ethane product stream, and optionally a propane product stream. The second stream in the light ends splitter bottoms line 166 may be optionally separated in another depropanizer column 170 to produce a depropanizer overhead product stream rich in propane in the depropanizer overhead line 172 and a depropanizer bottoms stream rich in C4+ hydrocarbons in depropanizer bottoms line 174. However, the second depropanizer column may be omitted when propane is recycled to the NEP reactor and in such an embodiment, no depropanized bottoms product stream is produced.

The first light ends net overhead vapor stream comprising light ends is produced from the receiver 132 in the receiver overhead line 136 and passed to a second fractionation column which is a demethanizer fractionation column 140. The net first light ends overhead liquid stream in line 138 is also passed to the demethanizer column 140. The net first light ends overhead liquid stream in line 138 may be passed at a location below the location at which the first light ends net overhead vapor stream in line 136 is passed to the demethanizer fractionation column 140. The demethanizer fractionation column 140 may be operated at an overhead pressure of about 1241 kPa (gauge) (180 psig) to about 2068 kPa (gauge) (300 psig), and a bottoms temperature of about −30° C. (−86° F.) to about 40° C. (104° F.). The demethanizer fractionation column 140 separates the light hydrocarbons such as methane and hydrogen in a demethanizer overhead stream which is produced in a demethanizer overhead line 142. A demethanized bottoms stream comprising C2+ hydrocarbons is produced in bottoms line 154 from the demethanizer fractionation column 140. Optionally, a demethanizer first side stream may be taken in a demethanizer side line 144 from the demethanizer fractionation column 140. The demethanizer first side stream in line 144 may comprise ethane and propane. The demethanizer first side stream in line 144 may be rich in C2+ paraffins. The demethanizer bottoms stream comprising C2+ hydrocarbons in line 154 may be recovered as a C2 product stream. The demethanizer first side stream in line 144 may be employed in an embodiment that includes a deethanizer column 160. In an aspect, the demethanizer first side stream in line 144 is a liquid stream. In an embodiment, the demethanizer bottoms stream comprising C2+ paraffins in line 154 and optionally the demethanizer first side stream in line 144 may be further separated to provide an ethane product stream.

An optional deethanizer column 160 is disclosed for separating ethane from the demethanizer bottoms stream in line 154. In an aspect, the demethanizer bottoms stream in line 154 may be passed to a demethanizer bottoms exchanger 156 to heat the demethanizer bottoms stream and provide a heated demethanizer bottoms stream in line 158 which is passed to the deethanizer column 160. The optional demethanizer first side stream in line 144 may also be passed to the deethanizer column 160. The demethanizer first side stream in line 144 may be passed to a demethanizer sidedraw exchanger 146 to heat the demethanizer first side stream and provide a heated demethanizer first side stream in line 148 which is passed to the deethanizer column 160. In an aspect, the demethanizer bottoms stream in line 154 and the optional demethanizer first side stream in line 144 may be heat exchanged with the first compressed stream in line 126 in the heat exchanger 128 to provide cooling to the first compressed stream. The heated first side stream in line 148 and the heated demethanizer bottoms stream in line 158 may be taken from the heat exchanger 128 and passed to the deethanizer column 160. In this embodiment, heat exchangers 128, 146 and 156 may be one or more heat exchangers in series with the demethanized bottoms stream in line 154 on one side of the heat exchanger and the first compressed stream on the other side of the heat exchanger and optionally the demethanizer first side stream in line 144 on one side of the heat exchanger and the first compressed stream on the other side of the heat exchanger. In an embodiment, heat exchangers 128, 146, and 156 may be a single multi-sided heat exchanger with the first compressed stream in line 126 on one side exchanging heat with the demethanized bottoms stream in line 154 on another side and the demethanizer side stream in line 144 on an additional other side. This heat exchanger may be constructed of brazed aluminum, carbon steel, or 300 series stainless steel, and it may be of plate and frame, plate and fin, or spiral wound tube construction. In an embodiment, the heated demethanizer first side stream in line 148 is passed to a side column inlet at a higher location in the deethanizer column 160 than a bottoms column inlet in the deethanizer column for the heated demethanizer bottoms stream in line 158.

The deethanizer column 160 produces a deethanizer overhead product stream rich in ethane in the overhead line 162. An overhead ethane product stream may be provided in the overhead line 162 from the deethanizer column 160. The deethanizer column 160 may be operated at an overhead pressure of about 1241 kPa (gauge) (180 psig) to about 2068 kPa (gauge) (300 psig), and a bottoms temperature of about 40° C. (104° F.) to about 100° C. (212° F.). A deethanized bottoms stream comprising propane may be taken in a bottoms line 164 from the deethanizer column 160.

A deethanizer reboiler stream may be taken in line 165 from the deethanized bottoms stream in line 164. In an aspect, the splitter light stream in line 116 may be heat exchanged with the deethanized reboiler stream in line 165. The deethanizer reboiler stream in line 165 may be heated by heat exchange with the splitter light stream in the splitter overhead line 116 in a deethanizer reboiler 167 to provide the cooled splitter light stream in line 118 and a deethanized reboiled stream in line 169. The heated reboiled stream in line 169 is passed back to the bottom of the deethanizer column 160. A net deethanized bottoms stream is provided in line 171 rich in propane.

In an alternate embodiment, the deethanizer reboiler 167 may comprise a stab-in reboiler. In this alternate embodiment, the deethanizer reboiler 167 is incorporated directly in the deethanizer column 160. In this alternate embodiment, the splitter light stream in the light ends splitter overhead line 116 may be heat exchanged directly with liquid in column 160 in the deethanizer reboiler 167 to provide the cooled light ends splitter stream in line 118.

In an aspect, the overhead ethane product stream in line 162 may be charged to an ethylene producing unit 195 in which ethane in the ethane stream is converted into ethylene. In an embodiment, the ethylene producing unit 195 may include a steam cracking unit. The overhead ethane product stream in line 162 may be cracked under steam in a furnace to produce a cracked stream including an ethylene stream in line 196. The overhead ethane product stream in line 162 may be charged to the ethane steam cracking unit in the gas phase. The ethane steam cracking unit 195 may preferably be operated at a temperature of about 750° C. (1382° F.) to about 950° C. (1742° F.). The cracked stream exiting the furnace of the ethane steam cracking unit 195 may be in a superheated state. One or more quench columns, or other devices but preferably an oil quench column and/or a water quench column, may be used for quenching or separating the cracked stream into a plurality of cracked streams. The ethane steam cracking unit 195 may further comprise additional distillation columns, amine wash columns, compressors, expanders, etc. to separate the cracked stream into cracked streams rich in individual light olefins, the most predominant of which is the ethylene stream in line 196. The ethylene stream in line 196 may comprise a yield of at least 75 wt %, preferably at least 80 wt %, ethylene based on the overhead ethane product stream in line 162. Among the other components in the cracked stream exiting the ethane steam cracking unit 195 may be hydrogen, methane, propylene, butene, and pyrolysis gas. Each of these components may be recovered and further processed.

The ethylene stream in line 196 and perhaps a propylene stream from the ethylene producing unit may be recovered or transported to polymerization plants, chemical plants or exported. A butene stream may be recovered and used to produce plastics or other petrochemicals by processes such as polymerization or exported. Product recovery of at least 50 wt %, typically at least 60 wt % and suitably at least 70 wt % of valuable ethylene, propylene, and butylene products is achievable from the ethane steam cracking unit 195 based on the overhead ethane product stream in line 162.

The present disclosure provides an optional depropanizer column 170 for separating a propane product stream from the light ends bottoms stream in line 166. The depropanizer column 170 may be deemed a second depropanizer column if the light ends splitter column 120 is deemed a first depropanizer column. A depropanizer overhead stream comprising propane is separated in an overhead line 172 in the depropanizer column 170. A depropanized bottoms stream rich in C4+ paraffins is separated in a depropanizer bottoms line 174 from the depropanizer column 170. The depropanized overhead stream in line 172 may be combined with the net deethanized bottoms stream in line 171 to provide a propane product stream in line 182.

In an aspect, the propane product stream in line 182 may be charged to a propylene producing unit 185 in which propane in the propane product stream is converted into propylene. The propylene producing unit 185 may be a propane dehydrogenation (PDH) unit. PDH catalyst is used in a dehydrogenation reaction process to catalyze the dehydrogenation of propane. The conditions in the dehydrogenation reactor may include a temperature of about 500 to about 800° C., a pressure of about 40 to about 310 kPa (abs) and a catalyst to oil ratio of about 5 to about 100.

The dehydrogenation reaction may be conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated in a regenerator. The regenerator may combust coke from the dehydrogenation catalyst and fuel gas to ensure sufficient enthalpy in the dehydrogenation reactor to promote the endothermic reaction.

The dehydrogenation catalyst selected should minimize cracking reactions and favor dehydrogenation reactions. Suitable catalysts for use herein include an active metal which may be dispersed in a porous inorganic carrier material such as silica, alumina, silica alumina, zirconia, or clay. An exemplary embodiment of a catalyst includes alumina or silica-alumina containing gallium, a noble metal, and an alkali or alkaline earth metal.

The catalyst support comprises a carrier material, a binder and an optional filler material to provide physical strength and integrity. The carrier material may include alumina or silica-alumina. Silica sol or alumina sol may be used as the binder. The alumina or silica-alumina generally contains alumina of gamma, theta and/or delta phases. The catalyst support particles may have a nominal diameter of about 400 to about 5000 micrometers with the average diameter of about 600 to about 3500 micrometers. Preferably, the surface area of the catalyst support is about 85 to about 140 m2/g.

The fluidized dehydrogenation catalyst may comprise a dehydrogenation metal on a support. The dehydrogenation metal may be a one or a combination of transition metals. A noble metal may be a preferred dehydrogenation metal such as platinum or palladium. Gallium is an effective metal for paraffin dehydrogenation. Metals may be deposited on the catalyst support by impregnation or other suitable methods or included in the carrier material or binder during catalyst preparation.

The acid function of the catalyst should be minimized to prevent cracking and favor dehydrogenation. Alkali metals and alkaline earth metals may also be included in the catalyst to attenuate the acidity of the catalyst. Rare earth metals may be included in the catalyst to control the activity of the catalyst. Concentrations of 0.001% to 10 wt % metals may be incorporated into the dehydrogenation catalyst. In the case of the noble metals, it is preferred to use about 10 parts per million (ppm) by weight to about 600 ppm by weight noble metal. More preferably it is preferred to use about 10 to about 100 ppm by weight noble metal. The preferred noble metal is platinum. Gallium should be present in the range of 0.3 wt % to about 3 wt %, preferably about 0.5 wt % to about 2 wt %. Alkali and alkaline earth metals may be present in the range of about 0.05 wt % to about 1 wt %.

Regenerated catalyst may be contacted with the propane stream perhaps with a fluidizing gas to lift the propane stream and dehydrogenation catalyst up a riser while dehydrogenation occurs. Above the riser spent dehydrogenation catalyst and propylene product may be separated by a centripetal separation device. Propylene product gas may be quenched with a cooling fluid to prevent over reaction to undesired by-products. Separation of the propylene product from the PDH effluent stream may include quench contacting and fractionation to produce a propylene product stream in line 186. Unreacted propane may be recycled to the dehydrogenation reactor and lighter gases may be recycled to the regenerator as fuel gas to be combusted to provide enthalpy for the reaction.

The propylene producing unit 185 may also employ a catalytic moving bed reactor. The reactor section may comprise several radial flow reactors in parallel or series heated by charge and interstage heaters. The propane stream perhaps with added hydrogen flows in each dehydrogenation reactor from a screened center pipe through an annular dehydrogenation catalyst bed to an outer effluent annulus. Flow may be in the reverse fashion. The dehydrogenation catalyst may comprise a noble metal or mixtures thereof, a modifier selected from the group consisting of alkali metals or alkaline-earth metals and mixtures thereof, a component selected from the group consisting of tin, germanium, lead, indium, gallium, thallium, and mixtures thereof, and a porous support forming a catalyst particle. The catalyst support may comprise oil dropped alumina spheres.

Dehydrogenation conditions may include a temperature of from about 400 to about 900° C., a pressure of from about 0.01 to 10 atmospheres absolute, and a liquid hourly space velocity (LHSV) of from about 0.1 to 100 hr-1. The pressure in the dehydrogenation reactor is maintained as low as practicable, consistent with equipment limitations, to maximize chemical equilibrium advantages. Spent dehydrogenation catalyst in the annular catalyst bed may be withdrawn from the bottom of the bed, forwarded to a regenerator to combust coke from the catalyst with air at about 450 to about 600° C. Noble metal on the catalyst may be redispersed by an oxyhalogenation process, dried and returned to the top of the dehydrogenation catalyst bed as regenerated dehydrogenation catalyst.

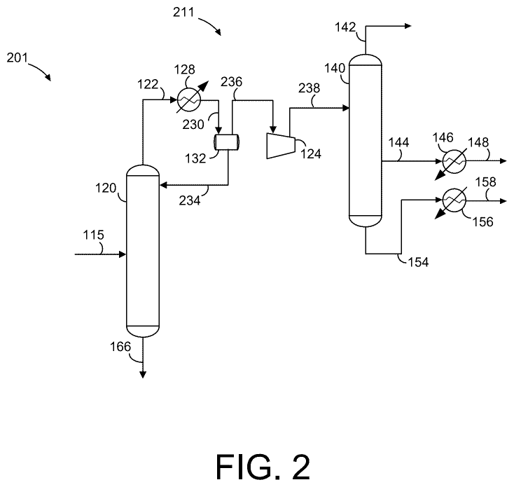

Dehydrogenation effluent from the propylene producing unit 185 may be cooled, compressed, dried and hydrogen is cryogenically separated from the hydrocarbons with a net gas purity of 85 to 93 mol % hydrogen. Hydrocarbon liquid is selectively hydrogenated to convert diolefins and acetylenes and the hydrocarbon liquid is fractionated in a deethanizer column to remove ethane and propylene is split from propane in a propane-propylene splitter column to provide polymer-grade propylene in line 186. Propane may be recycled as feed to the propylene producing unit 185. FIG. 2 illustrates another embodiment 201 of a process and an apparatus for separating paraffins. In the embodiment as shown in FIG. 2, the first stream comprising C3− hydrocarbons is cooled and separated before compressing it in the compressor 124 of the separation unit 211. Elements in FIG. 2 with the same configuration as in FIG. 1 will have the same reference numeral as in FIG. 1. The configuration and operation of the embodiment of FIG. 2 is essentially the same as in FIG. 1 with the following exceptions.

As shown in FIG. 2, the splitter net overhead vapor stream in line 115 from FIG. 1 may be passed to the light ends splitter column 120. C1-C4 hydrocarbons may be separated in the light ends splitter column 120. A first overhead stream comprising C3− hydrocarbons may be taken from an overhead in a total overhead line 122 from the light ends splitter column 120. A second stream comprising C3+ hydrocarbons may be taken from a bottom in a bottoms line 166 from the light ends splitter column 120. More than about 99 mol % of C2− hydrocarbons may be recovered in the light ends splitter overhead stream of the light ends splitter column overhead line 136, and more than about 95 mol % of C4+ hydrocarbons may be recovered in the light ends splitter bottoms stream in the light ends splitter bottoms line 166.

The first overhead stream in line 122 may be passed to a light ends splitter condenser 128 to cool and partially condense the first stream in line 122. A cooled first stream may be provided in line 230 from the light ends splitter condenser 128. The light ends splitter condenser 128 may comprise one or more heat exchangers. Suitable heat exchange media for condensing the first overhead stream in line 122 in the light ends splitter condenser 128 include air, cooling water, refrigerants such as propane or propylene, and other process streams. The cooled first stream in line 230 may be passed to the light ends splitter receiver 132 for separation into a second light ends net overhead vapor stream in line 236 and a second light ends overhead liquid stream in line 234. In an aspect, an entirety of the second light ends overhead liquid stream in line 234 may be passed to the light ends splitter column 120.

The second light ends net overhead vapor stream in line 236 may be compressed in the light ends compressor 124 to provide a second compressed stream in line 238. In an embodiment, the second light ends net overhead vapor stream in line 236 is compressed to a pressure of at least about 2.1 MPa (g) (300 psig) or at least about 2.8 MPa (g) (400 psig) or at least about 3.1 MPa (g) (450 psig) to provide a second compressed stream in line 238. The second compressed stream in line 238 may be passed to the demethanizer column 140 as described for FIG. 1. The rest of the process is same as previously described in FIG. 1.

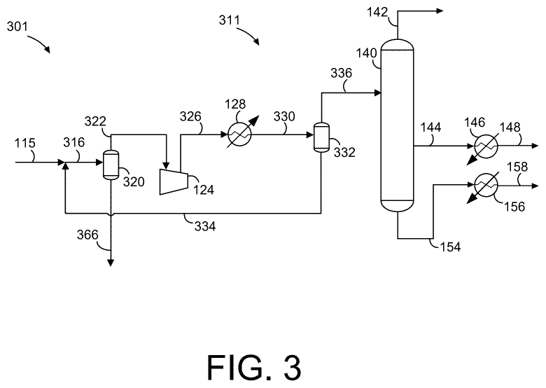

Another embodiment 301 of a process and an apparatus for separating paraffins is illustrated in FIG. 3. In the embodiment as shown in FIG. 3, the separation unit 311 comprises a first separator 320 and a second separator 332. Elements in FIG. 3 with the same configuration as in FIG. 1 will have the same reference numeral as in FIG. 1. The configuration and operation of the embodiment of FIG. 3 is essentially the same as in FIG. 1 with the following exceptions.

A splitter net overhead vapor stream is produced in line 115 from the splitter receiver as shown in FIG. 1. The splitter net overhead vapor stream in line 115 is passed the separation unit 311. As described later in detail, the splitter net overhead vapor stream in line 115 may be combined with a recycle liquid stream in line 334 to provide a combined stream in line 316 which is separated in the separation unit 311.

In the embodiment as shown in FIG. 3, the combined stream in line 316 may be passed to a first separator 320. In the first separator 320, C3− hydrocarbons are separated in a first separated vapor stream which may be produced in the overhead in line 322. C3+ hydrocarbons may be produced in a first bottom liquid stream in a bottoms line 366 from the first separator 320. The first separator 320 may be operated at a pressure of about 1034 kPa (gauge) (150 psig) to about 1724 kPa (gauge) (250 psig), and a temperature of about 20° C. (68° F.) to about 50° C. (122° F.). In an exemplary embodiment, the first separator 320 may be a first knock out drum (KOD).

The first separated vapor stream in line 322 may be compressed in the light ends compressor 124 to provide a first compressed stream in line 326. In an exemplary embodiment, the first separated stream in line 322 may be compressed to a pressure of about 1724 kPa (gauge) (250 psig) to about 3792 kPa (gauge) (550 psig) in the light ends compressor 124. In an embodiment, the first separated vapor stream in line 322 is compressed to a pressure of at least about 2.1 MPa (g) (300 psig) or at least about 2.8 MPa (g) (400 psig) or at least about 3.1 MPa (g) (450 psig) to provide a first compressed stream in line 326.

The first compressed stream in line 326 may be passed to the light ends splitter condenser 128 to partially condense the first compressed stream in line 326 and provide a first cooled stream in line 330. The light ends splitter condenser 128 may comprise one or more heat exchangers. Suitable heat exchange media for condensing the first compressed stream in line 326 in the light ends splitter condenser 128 include air, cooling water, refrigerants such as propane or propylene, and other process streams. In an exemplary embodiment, the first compressed stream in line 326 may be cooled to a temperature of about 30° C. (86° F.) to about 50° C. (122° F.) in the light ends splitter condenser 128. The first cooled stream in line 330 may be passed to a second separator 332 for separating the light ends such as C3− hydrocarbons.

In second separator 332, the first cooled stream in line 330 is separated into a second separated vapor stream comprising C3− hydrocarbons in line 336. The second bottom liquid stream in line 334 is taken from the bottom of the second separator 332 and recycled to the first separator 320. The second bottom liquid stream in line 334 may be combined with the splitter net overhead vapor stream in line 115 and recycled to the first KOD 320 as earlier described. The second separator 332 may be operated at a pressure of about 1724 kPa (gauge) (250 psig) to about 3792 kPa (gauge) (550 psig), and a temperature of about 30° C. (86° F.) to about 50° C. (122° F.). In an exemplary embodiment, the second separator 332 may be a second KOD.

The second separated vapor stream in line 336 is passed to the second fractionation column 140 which may be a demethanizer fractionation column 140. The demethanizer fractionation column 140 may be operated at an overhead pressure of about 1241 kPa (gauge) (180 psig) to about 2068 kPa (gauge) (300 psig), and a bottoms temperature of about −30° C. (−22° F.) to about 40° C. (104° F.). The demethanizer fractionation column 140 separates the light hydrocarbons such as methane and hydrogen in a demethanizer overhead stream which is produced in a demethanizer overhead stream in line 142 from the demethanizer fractionation column 140. A demethanized bottoms stream comprising C2+ hydrocarbons is produced in a bottoms line 154 from the demethanizer fractionation column 140. Optionally, a first side stream may be taken in a demethanizer side line 144 from the demethanizer fractionation column 140. The first side stream in the demethanizer side line 144 and the demethanized bottoms stream in the bottoms line 154 may be cooled and separated in the deethanizer column 160 of FIG. 1. The rest of the process is the same as previously described in FIG. 1.

### EXAMPLES

### Example 1

The splitter column 110 of FIG. 1 was simulated using process modeling software. Table 1 provides simulated stream properties and compositions for the light paraffinic stream in line 102.

TABLE 1

| | |
|---|---|
| Stream | 102 |
| Temperature (° C.) | 130 |
| Pressure (kPa(a)) | 1712 |
| Vapor Fraction | 0.956 |
| mass flow (kg/h) | 100,000 |
| Composition (wt %) | |
| Hydrogen | 1.0% |
| Methane | 5.0% |
| Ethane | 27.0% |
| Propane | 27.0% |
| n-Butane | 5.0% |
| Toluene | 32.0% |
| Naphthalene | 3.0% |

The reactor effluent stream in line 102 was fed to a trayed distillation column 110. The splitter light stream in line 116 was cooled to about 40° C. in the splitter condenser 117 then passed to the splitter receiver 119 to separate the splitter net overhead vapor stream comprising C4− in line 115 and the splitter overhead liquid stream in line 121 which was returned to the top of the column. All column liquid was collected below the feed stage, heated to 197° C. in an upper reboiler 113, and returned to the column at the tray below the draw stage. The splitter side stream was drawn from the upper reboiler return tray. A portion of the column bottoms in line 195 were reboiled in a lower reboiler 197 and returned to the column in a lower reboiled stream in line 198. The net liquid bottoms stream is taken as the splitter heavy stream in line 194. The splitter condenser 117 duty for this separation was −21.3 GJ/h which may be provided by cooling water or another suitable cooling medium. The upper reboiler duty was 5.2 GJ/h, and the lower reboiler duty was 1.1 GJ/h. The splitter net overhead vapor stream in line 115 is a suitable feed to the separation unit described in following examples.

### Example 2

The paraffins separation process 311 illustrated in FIG. 3 was simulated using process modeling software. The splitter net overhead vapor stream of Example 1 was used as the light paraffins stream in line 115 which was fed to the process 301. The light paraffins stream in line 115 was combined with a recycle liquid stream in line 334 and passed to a compressor suction separator 320 to separate a first vapor product stream in line 322 and a liquid product stream in line 366 comprising predominantly C3+ hydrocarbons. The first vapor product in line 322 was passed to a compressor 124 and compressed to 3503 kPa (gauge) (508 psig) to provide a compressed vapor product stream in line 326. The compressed vapor product stream was passed to a compressor discharge cooler 328 and cooled to 40° C. to provide a cooled compressed vapor product stream in line 330. The cooled compressed vapor product stream was passed to a compressor discharge separator 332 to provide a second vapor product stream in line 336 comprising predominately C3− hydrocarbons and the recycle liquid stream in line 334. The second vapor product stream is passed to a demethanizer column 140 to provide a demethanizer overhead stream in line 142, a demethanizer side stream in line 144, and a demethanizer bottoms stream in line 154. The compressor 124 process work was 1,860 kW to compress the first vapor product to 508 psig. The second vapor product stream in line 336 processed in the demethanizer column had a significant amount of C4+ hydrocarbons of about 7.5 wt % which increases the stream viscosity and demethanizer reboiler temperature.

### Example 3

The paraffins separation process 201 illustrated in FIG. 2 was simulated using process modeling software. Table 3 provides simulated stream properties and compositions for select streams. The splitter net overhead vapor stream of Example 1 was used as the light paraffins stream in line 115 which was passed to the light ends splitter column 120. The light ends splitter total overhead stream in line 122 was condensed in condenser 128 at −4° C. and separated into liquid reflux second light ends overhead liquid stream in line 234 and second light ends net overhead vapor stream in line 236 in light ends splitter receiver 132. The second light ends net overhead vapor stream in line 236 was compressed to 3447 kPa (gauge) (500 psig) in light ends compressor 124 and passed to demethanizer column 140 to produce the demethanizer overhead stream in line 142, the demethanizer side stream in line 144, and the demethanizer bottoms stream in line 154. The light ends splitter condenser 128 duty was −10.1 GJ/h. The light ends compressor 124 process work is 1,380 kW to compress the second light ends net overhead vapor stream in line 236 to 500 psig.

Compared with Example 2, less than 5% of the C4 hydrocarbons and no C5+ hydrocarbons were passed to the demethanizer column to be fractionated resulting in 13% less feed to the demethanizer column by mass. This was due to better separation efficiency in the light ends splitter column 120 than can be achieved with a series of separator vessels as in Example 2. As a result, the demethanizer reboiler temperature decreased by 5° C. accompanied by a reduction in reboiler duty. The reduced feed rate also decreased the size of the demethanizer column and associated heat exchange equipment, and it reduces the refrigerant duty required to cool the feed and provide condenser duty to drive the separation. Another benefit of the paraffins separation process 201 in Example 3 is that the compressor work required to compress the demethanizer feed is 26% lower than in Example 2. The demethanizer side stream in line 144 may be heat exchanged with the light ends splitter total overhead stream to provide condensing duty for the light ends splitter condenser 128. Heating the demethanizer side stream to −8° C. to provide sufficient approach temperature for heat exchange provides 0.5 GJ/h duty which is 5% of the total condensing duty required in light ends splitter condenser 128.

### Example 4

The paraffins separation process and apparatus 101 illustrated in FIG. 1 was simulated using process modeling software. In the simulation, the splitter net overhead vapor stream in line 115 was passed to the light ends splitter column 120. The first stream in the light ends splitter total overhead line 122 was compressed in light ends compressor 124, condensed in light ends splitter condenser 128 at 23° C., and separated into liquid reflux stream in line 134 and the first light ends net overhead vapor stream in line 136 in light ends splitter receiver 132. The first light ends net overhead vapor stream in line 136 was passed to the demethanizer column 140 to produce the demethanizer overhead stream in line 142, the demethanizer side stream in line 144, and the demethanizer bottoms stream in line 154. The light ends splitter condenser 128 duty was −16.5 GJ/h. The light ends compressor 124 process work was 2,075 kW to compress the light ends total overhead vapor stream to 500 psig.

The paraffins separation process and apparatus 101 of Example 4 demonstrates similar benefits over Example 2 with respect to the demethanizer column. The light ends splitter net overhead stream fed to the demethanizer column was 13% smaller by mass with less than 5% of the C4 hydrocarbons and no C5+ hydrocarbons. The reduced feed rate and significantly less C4+ hydrocarbons resulted in a smaller demethanizer column and heat exchange equipment, lower reboiler temperature, lower reboiler duty, and less refrigerant duty required to cool the demethanizer feed stream and provide condenser duty to drive the separation. A difference from Example 3 is that the separations process and apparatus 101 compresses the light ends splitter total overhead stream rather than the light ends splitter net overhead stream. The light ends compressor process work for separation process and apparatus 101 of Example 4 was 50% higher than for separation process 201 of Example 3 because more gas was compressed, but a benefit is that the light ends splitter condenser 128 was operated at a higher temperature of 23° C. compared to −4° C. in Example 3. This allowed for higher temperature utilities to be used for condensing duty, or it can enable better heat integration with other process streams such as the demethanizer side stream in line 144. The demethanizer side stream in line 144 may be heat exchanged with the light ends splitter total overhead stream to provide condensing duty for the light ends splitter condenser 128. Heating the demethanizer side stream to 15° C. to provide sufficient approach temperature for heat exchange provided 7.0 GJ/h duty which is 42% of the total condensing duty required in light ends splitter condenser 128.

### Specific Embodiments

While the following is described in conjunction with specific embodiments, it will be understood that this description is intended to illustrate and not limit the scope of the preceding description and the appended claims.

A first embodiment of the present disclosure is a process for separating paraffins comprising separating a paraffinic stream to provide a first stream comprising C3− hydrocarbons and a second stream comprising C3+ hydrocarbons; compressing the first stream to provide a first compressed stream; and fractionating the first compressed stream in a fractionation column to provide an overhead stream and a bottom stream comprising C2+ hydrocarbons. An embodiment of the present disclosure is one, any or all of prior embodiments in this paragraph up through the first embodiment in this paragraph further comprising cooling the first compressed stream to provide a first cooled stream; separating the first cooled stream to provide a first vapor stream and a first liquid stream; and passing the first vapor stream to the fractionation column. An embodiment of the present disclosure is one, any or all of prior embodiments in this paragraph up through the first embodiment in this paragraph further comprising passing a portion of the first c liquid stream to the fractionation column. An embodiment of the present disclosure is one, any or all of prior embodiments in this paragraph up through the first embodiment in this paragraph further comprising passing the second stream to a depropanizer column to provide an overhead stream comprising propane and a depropanized bottom stream comprising C4+ hydrocarbons. An embodiment of the present disclosure is one, any or all of prior embodiments in this paragraph up through the first embodiment in this paragraph further comprising passing the bottom stream comprising C2+ hydrocarbons to a deethanizer column to provide an overhead product stream comprising ethane, and a deethanized bottoms stream comprising propane; and combining the deethanized bottoms stream with the overhead stream comprising propane to provide a propane product stream. An embodiment of the present disclosure is one, any or all of prior embodiments in this paragraph up through the first embodiment in this paragraph further comprising fractionating a reactor effluent stream in a splitter column to separate aromatics in a splitter heavy stream and provide a splitter light stream comprising paraffins; and taking the paraffinic stream from the splitter light stream. An embodiment of the present disclosure is one, any or all of prior embodiments in this paragraph up through the first embodiment in this paragraph further comprising contacting a naphtha stream with a catalyst in the presence of hydrogen to produce paraffins; and taking the reactor effluent stream from the paraffins. An embodiment of the present disclosure is one, any or all of prior embodiments in this paragraph up through the first embodiment in this paragraph further comprising recycling the splitter heavy stream to the contacting step. An embodiment of the present disclosure is one, any or all of prior embodiments in this paragraph up through the first embodiment in this paragraph further comprising heat exchanging the first compressed stream with the bottom stream comprising C2+ hydrocarbons to cool the first compressed stream and heat the bottom stream comprising C2+ hydrocarbons. An embodiment of the present disclosure is one, any or all of prior embodiments in this paragraph up through the first embodiment in this paragraph further comprising taking a first side stream from the fractionation column; and fractionating the first side stream in the deethanizer column. An embodiment of the present disclosure is one, any or all of prior embodiments in this paragraph up through the first embodiment in this paragraph further comprising heat exchanging the first compressed stream with the first side stream to cool the first compressed stream and heat the first side stream. An embodiment of the present disclosure is one, any or all of prior embodiments in this paragraph up through the first embodiment in this paragraph further comprising heat exchanging the first compressed stream with the bottom stream comprising C2+ hydrocarbons and the first side stream to cool the first compressed stream and heat the bottom stream comprising C2+ hydrocarbons and the first side stream. An embodiment of the present disclosure is one, any or all of prior embodiments in this paragraph up through the first embodiment in this paragraph further comprising heat exchanging the splitter light stream with a deethanized bottoms stream in a deethanizer reboiler to cool the splitter light stream and provide a cooled splitter light stream; and separating paraffins from the cooled splitter light stream. An embodiment of the present disclosure is one, any or all of prior embodiments in this paragraph up through the first embodiment in this paragraph further comprising separating the cooled splitter light stream in a splitter receiver into a splitter overhead vapor stream and a splitter overhead liquid stream; fractionating the splitter overhead liquid stream in the splitter column; and taking the paraffinic stream from the splitter overhead vapor stream. An embodiment of the present disclosure is one, any or all of prior embodiments in this paragraph up through the first embodiment in this paragraph further comprising separating the paraffinic stream in a first fractionation column to provide the first stream and the second stream; and fractionating the first compressed stream in a second fractionation column to provide the overhead stream and the bottom stream comprising C2+ hydrocarbons. An embodiment of the present disclosure is one, any or all of prior embodiments in this paragraph up through the first embodiment in this paragraph further comprising cooling the first stream to provide a cooled first stream; separating the cooled first stream to provide a second vapor stream and a second liquid stream; and passing the second vapor stream to the fractionation column. An embodiment of the present disclosure is one, any or all of prior embodiments in this paragraph up through the first embodiment in this paragraph further comprising compressing the second vapor stream to provide a second compressed stream; and passing the second compressed stream to the fractionation column.

A second embodiment of the present disclosure is a process for producing paraffins comprising contacting a naphtha stream with a catalyst in the presence of hydrogen to produce paraffins; taking a paraffinic stream from the paraffins; separating the paraffinic stream to provide a first stream comprising C3− hydrocarbons and a second stream comprising C3+ hydrocarbons; and fractionating the first stream in a fractionation column to provide an overhead stream and a bottoms stream comprising C2+ hydrocarbons. An embodiment of the present disclosure is one, any or all of prior embodiments in this paragraph up through the second embodiment in this paragraph further comprising fractionating a reactor effluent stream taken from the paraffins in a splitter column to provide a splitter heavy stream comprising aromatics and a splitter light stream comprising paraffins; and taking the paraffinic stream from the splitter light stream.

A third embodiment of the present disclosure is a process for producing paraffins comprising fractionating a paraffins stream in a splitter column to provide a splitter light stream comprising paraffins and a splitter heavy stream comprising aromatics; separating the splitter light stream to provide a first stream comprising C3− hydrocarbons and a second stream comprising C3+ hydrocarbons; and fractionating the first stream in a fractionation column to provide an overhead stream and a bottom stream comprising C2+ hydrocarbons.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for separating paraffins comprising:
separating a paraffinic stream to provide a first stream comprising C3− hydrocarbons and a second stream comprising C3+ hydrocarbons;
compressing said first stream to provide a first compressed stream; and
fractionating said first compressed stream in a fractionation column to provide an overhead stream and a bottom stream comprising C2+ hydrocarbons.

2. The process of claim 1 further comprising:
cooling said first compressed stream to provide a first cooled stream;
separating said first cooled stream to provide a first vapor stream and a first liquid stream; and
passing said first vapor stream to the fractionation column.

3. The process of claim 2 further comprising passing a portion of said first c liquid stream to the fractionation column.

4. The process of claim 2 further comprising:
passing said second stream to a depropanizer column to provide an overhead stream comprising propane and a depropanized bottom stream comprising C4+ hydrocarbons.

5. The process of claim 4 further comprising:
passing said bottom stream comprising C2+ hydrocarbons to a deethanizer column to provide an overhead product stream comprising ethane, and a deethanized bottoms stream comprising propane; and
combining said deethanized bottoms stream with said overhead stream comprising propane to provide a propane product stream.

6. The process of claim 1 further comprising:
fractionating a reactor effluent stream in a splitter column to separate aromatics in a splitter heavy stream and provide a splitter light stream comprising paraffins; and
taking said paraffinic stream from said splitter light stream.

7. The process of claim 6 further comprising:
contacting a naphtha stream with a catalyst in the presence of hydrogen to produce paraffins; and
taking said reactor effluent stream from said paraffins.

8. The process of claim 7 further comprising recycling said splitter heavy stream to said contacting step.

9. The process of claim 2, wherein cooling said first compressed stream to provide a first cooled stream comprises:
heat exchanging said first compressed stream with the bottom stream comprising C2+ hydrocarbons to cool said first compressed stream and heat said bottom stream comprising C2+ hydrocarbons.

10. The process of claim 5 further comprising:
taking a first side stream from the fractionation column; and
fractionating said first side stream in the deethanizer column.

11. The process of claim 10, wherein cooling said first compressed stream to provide a first cooled stream comprises:
heat exchanging the first compressed stream with said first side stream to cool said first compressed stream and heat said first side stream.

12. The process of claim 10, wherein cooling said first compressed stream to provide a first cooled stream comprises:
heat exchanging said first compressed stream with said bottom stream comprising C2+ hydrocarbons and said first side stream to cool said first compressed stream and heat said bottom stream comprising C2+ hydrocarbons and said first side stream.

13. The process of claim 6 further comprising:
heat exchanging said splitter light stream with a deethanized bottoms stream in a deethanizer reboiler to cool said splitter light stream and provide a cooled splitter light stream; and
separating paraffins from said cooled splitter light stream.

14. The process of claim 13, wherein the step of separating paraffins comprises:
separating said cooled splitter light stream in a splitter receiver into a splitter overhead vapor stream and a splitter overhead liquid stream;
fractionating said splitter overhead liquid stream in the splitter column; and
taking said paraffinic stream from said splitter overhead vapor stream.

15. The process of claim 1 further comprising:
separating said paraffinic stream in a first fractionation column to provide said first stream and said second stream; and
fractionating said first compressed stream in a second fractionation column to provide said overhead stream and said bottom stream comprising C2+ hydrocarbons.

16. The process of claim 1 further comprising:
cooling said first stream to provide a cooled first stream;
separating said cooled first stream to provide a second vapor stream and a second liquid stream; and
passing said second vapor stream to the fractionation column.

17. The process of claim 16 further comprising:
compressing said second vapor stream to provide a second compressed stream; and
passing said second compressed stream to the fractionation column.

18. A process for producing paraffins comprising:
contacting a naphtha stream with a catalyst in the presence of hydrogen to produce paraffins;
taking a paraffinic stream from said paraffins;
separating said paraffinic stream to provide a first stream comprising C3− hydrocarbons and a second stream comprising C3+ hydrocarbons; and
fractionating said first stream in a fractionation column to provide an overhead stream and a bottoms stream comprising C2+ hydrocarbons.

19. The process of claim 16 further comprising fractionating a reactor effluent stream taken from said paraffins in a splitter column to provide a splitter heavy stream comprising aromatics and a splitter light stream comprising paraffins; and taking said paraffinic stream from said splitter light stream.

20. A process for producing paraffins comprising:
fractionating a paraffins stream in a splitter column to provide a splitter light stream comprising paraffins and a splitter heavy stream comprising aromatics;
separating said splitter light stream to provide a first stream comprising C3− hydrocarbons and a second stream comprising C3+ hydrocarbons; and
fractionating said first stream in a fractionation column to provide an overhead stream and a bottom stream comprising C2+ hydrocarbons.
